(19) United States
(12) Patent Application Publication
(10) Pub. No.: US 2024/0285409 A1
(43) Pub. Date: Aug. 29, 2024

(54) PROSTHETIC ANCHORING COMPONENTS WITH INTERNAL VOIDS

(71) Applicant: Zimmer, Inc., Warsaw, IN (US)

(72) Inventors: Akshay Vinayak Dhamankar, Parsippany, NJ (US); Tim Yoko, Granger, IN (US); Brian D. Earl, South Bend, IN (US); Grant Szalewski, Warsaw, IN (US)

(21) Appl. No.: 18/584,483

(22) Filed: Feb. 22, 2024

Related U.S. Application Data
(60) Provisional application No. 63/447,738, filed on Feb. 23, 2023.

(51) Int. Cl. A61F 2/30; A61F 2/28

(52) U.S. Cl. CPC A61F 2/30771; A61F 2002/2892; A61F 2002/30841

(57) ABSTRACT

An anchoring component for implanting with a prosthetic component comprises an annular body comprising an interior surface defining a socket for receiving a stem of the prosthetic component and an exterior surface for engaging bone, an internal void within the annular body and a functional component disposed within the internal void to provide supplemental functionality to the anchoring component. The functional component can comprise a treatment fluid or a sensor module.

### CLAIM OF PRIORITY

This application claims the benefit of U.S. Provisional Patent Application Ser. No. 63/447,738, filed on Feb. 23, 2023, the benefit of priority of which is claimed hereby, and which is incorporated by reference herein in its entirety.

### TECHNICAL FIELD

This document pertains generally, but not by way of limitation, to prosthetic implant devices having stems configured to be inserted into bone. More specifically, but not by way of limitation, the present application relates to prosthetic anchoring components, such as sleeves and cones, that surround stems of tibial and femoral devices to facilitate attachment to bone when implanted.

### BACKGROUND

Prosthetic implant devices, such as femoral and tibial components, sometimes include a stem extending from a prosthetic component, such as a femoral condyle component or a tibial tray. The stem can be inserted into a bone to extend along a length of the diaphysis portion of the tibia, while the condyle or tray can be configured to abut a resected portion of the epiphysis portion of the femur or tibia. Sometimes the metaphysis portion of the bone below the epiphysis includes damaged or unhealthy cancellous bone at the resection. As such, it is sometimes desirable to remove weakened bone material, such as with a broach or reamer, to leave a space in the metaphyseal portion of the bone larger than the stem. Sometimes a sleeve or cone is positioned in the space around a stem for the tibial or femoral component in order to facilitate attachment of the prosthesis to the bone. A sleeve can attach to the stem, while a cone can be unattached to the stem with the space therebetween sometimes being filled with bone cement.

Examples of sleeves and cones for use with prosthetic implants are described in U.S. Pat. No. 8,721,733 to Bonitati; U.S. Pat. No. 11,172,940 to Servidio et al.; U.S. Pub. No. 2014/0277528 to Mines et al.; U.S. Pub. No. 2014/0277540 to Leszko et al.; and U.S. Pub. No. 2017/0000503 to Keefer et al.

### Overview

The present inventors have recognized, among other things, that problems to be solved in implanting prosthetic devices that utilize sleeves or cones can include the limited utility of many sleeves and cones. For example, sleeves typically comprise a conical body or a conical-like body that is elongated in the medial-lateral direction. Typically, these conical or conical-like bodies comprise pieces of material that are intended to occupy space within a bone previously occupied by diseased or damaged bone matter. The outer surface of the sleeve can engage healthy or cortical bone, while the inner surface of the sleeve can be configured to join to, or come close to, a stem of a tibial component. Thus, the sleeve can provide utility in providing firm attachment of the prosthetic tibial component to bone using the outer and inner surfaces of the sleeve. The space between the inner and outer surfaces is typically occupied by material of the sleeve. Thus, the sleeve provides little or no further benefit to the patient or prosthetic tibial component beyond the anchoring functionality. Additionally, the mass of material of the sleeve that is implanted into the patient can add weight to the prosthetic device, thereby potentially affecting the patient experience. Furthermore, the material of the sleeve between the inner and outer surfaces can add cost to the prosthetic device.

The present subject matter can help provide solutions to these problem, and other problems, by providing anchoring components, such as sleeves, cones and other structures configured to hold or anchor prosthetic components in engagement with bone matter, having internal cavities or voids that can reduce the weight and cost of the components and provide space within the components to allow for additional or supplemental functionality. For example, internal cavities or voids within a sleeve or cone can provide space for functional components, such as treatment fluids including drugs, biologics, bone-growth promoting material, antibiotic material, infection preventing mediums, bone autograft material, bone allograft material, and electronics, such as sensors and communications devices. The anchoring devices of the present disclosure can include porous structures that allow the treatment fluid to escape from the internal voids. As such, the internal void can comprise a macro-void that can hold a volume of treatment fluid, while a porous structure can include micro-voids that can allow the volume of treatment fluid to slowly escape, or elute, from the internal void by passing through the porous structure.

In an example, an anchoring component for implanting with a prosthetic component can comprise an annular body comprising an interior surface defining a socket for receiving a stem of the prosthetic component and an exterior surface for engaging bone, an internal void within the annular body and a functional component disposed within the internal void to provide supplemental functionality to the anchoring component.

In an example, the functional component can comprise a treatment fluid.

In an example, the functional component can comprise a sensor module.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

FIG. 1 is an exploded perspective view of a tibial component having a tibial stem with a sleeve configured to be disposed to surround the tibial stem housing.

FIG. 2 is a side cross-sectional view of a proximal end of a tibia having a reaming tool inserted into the metaphysis via the intramedullary canal of the tibia to form a reamed channel.

FIG. 3 is a side cross-sectional view of the proximal end of the tibia of FIG. 2 with the reaming tool removed and an epiphysis region of the bone resected at a resected surface.

FIG. 4 is a side cross-sectional view of the tibial component and sleeve of FIG. 1 inserted into the reamed intramedullary canal of FIG. 3 in a coupled configuration.

FIG. 5 is a front view of a first example of a sleeve having an internal void within an inner solid component that is surrounded by an outer porous component.

FIG. 6 is a top view of the sleeve of FIG. 5 showing a stem socket and the internal void connected to passages in phantom.

FIG. 7 is cross-sectional view of the sleeve of FIG. 5 taken at section 7-7 to show the internal void extending along a posterior side of the stem socket.

FIG. 8 is cross-sectional view of the sleeve of FIG. 6 taken at section 8-8 to show the internal void positioned on medial and lateral sides of the stem socket.

FIG. 9 is cross-sectional view of the sleeve of FIG. 7 taken at section 9-9 to show the internal void positioned on a posterior side of the stem socket.

FIG. 10 is cross-sectional view of the sleeve of FIG. 6 taken at section 10-10 to show one of the passages extending from an exterior of the sleeve to the internal void.

FIG. 11A is a perspective view of a plug that can be inserted into the passages of FIGS. 5, 6 and 10.

FIG. 11B is a cross-sectional side view of the plug lof FIG. 11A showing a tapered metering passage.

FIG. 12 is a perspective view of a second example of a sleeve having internal voids extending through an inner solid component that is surrounded by an outer porous component.

FIG. 13 is a top cross-sectional view of the sleeve of FIG. 12 taken at section 13-13 of FIG. 14 to show first and second internal voids extending within material of the inner solid component.

FIG. 14 is a front cross-sectional view of the sleeve of FIG. 13 taken at section 14-14 to show the first and second internal voids extending from a superior surface to an inferior surface.

FIG. 15 is a side cross-sectional view of the sleeve of FIG. 13 taken at section 15-15 to show an interface between the inner solid component and the outer porous component at anterior and posterior sides of the sleeve.

FIG. 16 is a perspective view of a third example of a sleeve having internal voids between an inner solid component and an outer porous component.

FIG. 17 is a cross-sectional view of the sleeve of FIG. 16 taken at section 17-17 showing first and second internal voids connected to first and second passages.

FIG. 18 is a front cross-sectional view of the sleeve of FIG. 16 taken at section 18-18 to show the first and second passages in the inner solid component.

FIG. 19 is a top cross-sectional view of the sleeve of FIG. 18 taken at section 19-19 to show the first and second passages extending between the inner solid component and the outer porous component.

FIG. 20 is a perspective view of a fourth example of a sleeve having internal voids between an inner solid component and an outer porous component that are connected by a central void.

FIG. 21 is a cross-sectional view of the sleeve of FIG. 20 showing upper and lower supports on the inner solid component for the outer porous component.

FIG. 22 is a cross-sectional view of the sleeve of FIG. 20 showing the internal void extending to an inferior surface of the sleeve.

FIG. 23 is a cross-sectional view of the sleeve of FIG. 20 showing the central void in fluid communication with the internal voids.

FIG. 24 is a schematic diagram of a membrane positioned between a porous component and a passage of an internal void of a solid component.

FIG. 25A is a close-up diagrammatic view of porous material suitable for use with the present disclosure comprising a plurality of ligaments forming open spaces.

FIG. 25B is a schematic cross-sectional view of one of the ligaments of FIG. 25A showing a carbon core surrounded by a film.

FIG. 26 is a schematic cross-sectional view of an exemplary sleeve of the present disclosure including additional functionality, including a sensor component and a tubing system.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

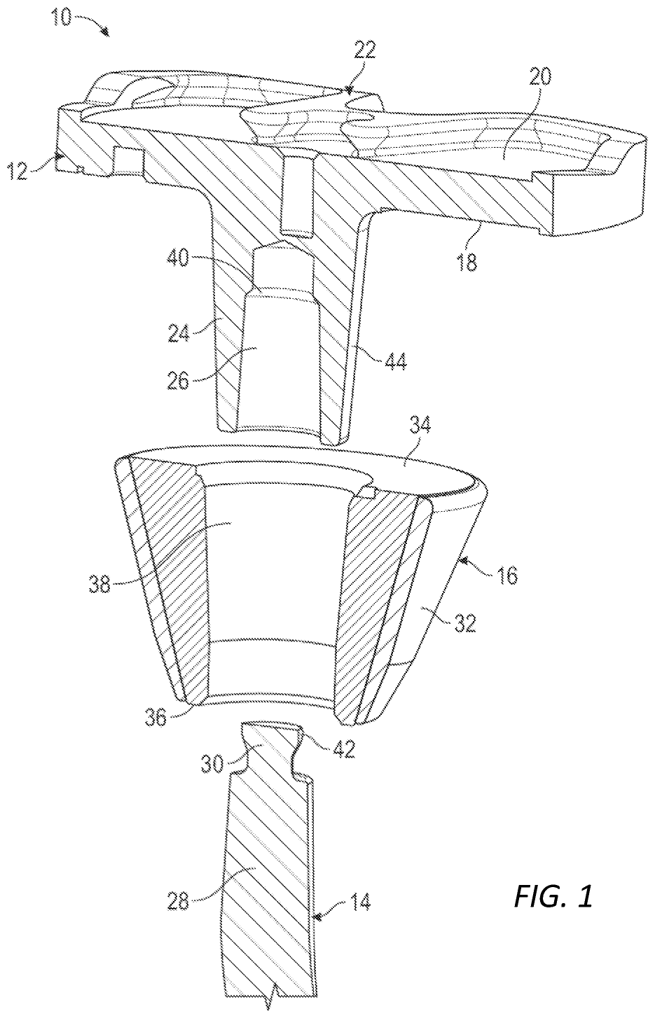

FIG. 1 is an exploded perspective view of tibial construct or tibial component 10 having tibial tray 12, tibial stem 14 and sleeve 16. Tibial tray 12 can comprise bone-facing surface 18, bearing surface 20, retaining features 22, stem housing 24 and stem housing socket 26. Tibial stem 14 can comprise shaft portion 28 and lockdown post 30. Sleeve 16 can comprise exterior surface 32, proximal portion 34, distal portion 36 and interior channel 38.

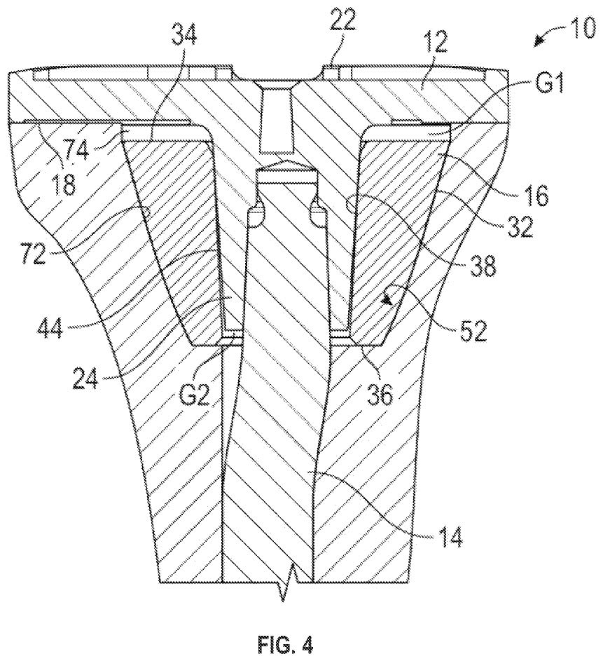

Tibial stem 14 is configured to be attached to tibial tray 12 and sleeve 16 is configured to surround tibial stem 14 and stem housing 24. Lockdown post 30 of tibial stem 14 can be inserted into stem housing socket 26 of tibial tray 12. Stem housing socket 26 can include lip 40 that can engage head 42 of lockdown post 30 to hold tibial stem 14 within stem housing socket 26. Outer surface 44 of stem housing 24 and interior channel 38 of sleeve 16 can be configured to engage each other to secure sleeve 16 to tibial tray 12. In examples, outer surface 44 can be configured to have a Morse taper and interior channel 38 can be configured to have a corresponding shape to seat on the Morse taper of outer surface 44, as shown in FIG. 4. Retaining features 22 can be used to secure various bearing components against bearing surface 20 of tibial component 10 to engage a femoral component. For example, retaining features 22 can include flanges having lips into which mating components of mobile or fixed bearings can be fitted to engage condylar surfaces of a femoral component.

Tibial stem 14 is configured to be pushed down into an intramedullary canal of a tibia bone to anchor tibial tray 12 so that bone-facing surface 18 contacts a resected bone surface of the tibia. Furthermore, sleeve 16 can be positioned around stem housing 24 to provide additional anchoring. For example, tibial stem 14 can be inserted into one or both of cancellous and cortical bone and sleeve 16 can be pushed into engagement with one or both of cancellous and cortical bone. Exterior surface 32 can be porous to promote bone in-growth, as is known in the art. The systems, devices and methods of the present disclosure can allow for the use of sleeves or cones that have internal cavities or voids that can provide space for functional components, such as treatment fluids including drugs, biologics, bone-growth promoting material, antibiotic material, infection preventing mediums, bone autograft material, bone allograft material, and electronics, including sensors and communications devices, as well as other types of functional components. The internal cavities or voids can additionally reduce the amount of material consumed by the sleeve or cone, thereby reducing cost and weight.

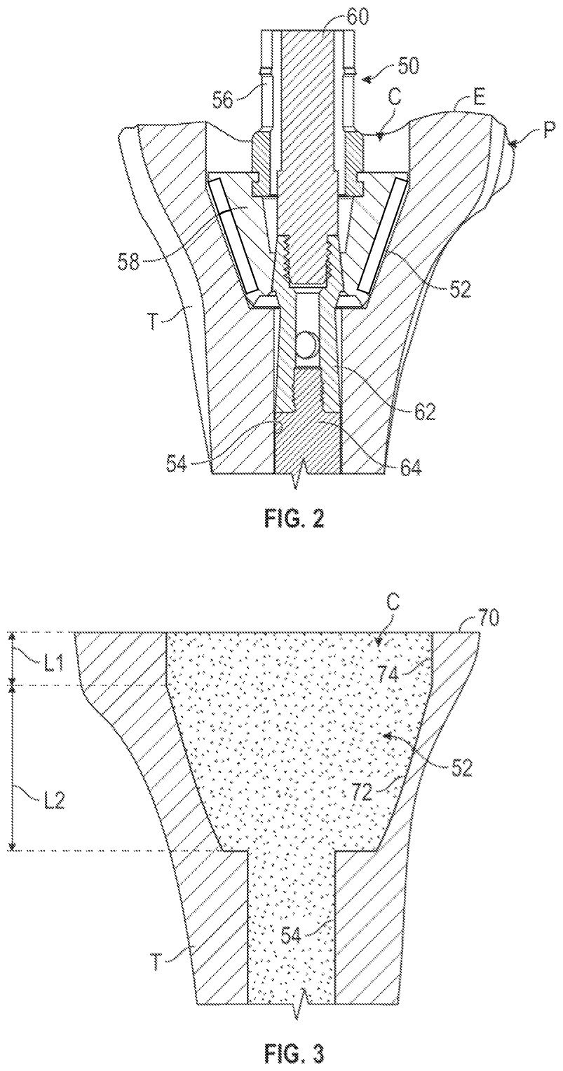

FIG. 2 is a side cross-sectional view of proximal end P of tibia T having reaming tool 50 inserted into metaphysis region of tibia T along an axis extending along intramedullary canal C of tibia T to form reaming channel 52. Reaming channel 52 can intersect stem channel 54, which can also extend along the axis of intramedullary canal C. Reaming tool 50 can comprise reamer shaft 56 and reaming head 58. Reamer shaft 56 and reaming head 58 can be cannulated to include an internal passage that receives stem extension post 60, which is connected to stem provisional 62 and extension post 64. Stem extension post 60 and extension post 64 can be co-axially aligned and fixed relative to each other. In other embodiments, stem provisional 62 and extension post 64 can be combined into a single piece.

With reaming head 58 inserted into tibia T, reamer shaft 56 can be reciprocated in an up-and-down motion relative to the orientation of FIG. 1 to widen reaming channel 52 along the axis of intramedullary canal C. As shown in FIG. 2, reaming head 58 can include various cutting surfaces, serrations, teeth, lands, edges or the like to chip away, cut away or otherwise remove bone. In embodiments, reaming head 58 can be inserted into reaming channel 52 to widen stem channel 54 into reaming channel 52. Stem channel 54 can be produced using a broach or a reamer in any suitable manner before or after reaming tool 50 is used to form reaming channel 52.

Stem channel 54 can comprise a generally cylindrical shaped passage extending longitudinally along an axis of tibia T. Stem channel 54 can extend into and through cancellous bone of tibia T. The cancellous bone of tibia T is surrounded by an outer layer of harder cortical bone. Stem channel 54 can form a passage for receiving a tibial post or stem that extends from a tibial component. For example, stem provisional 62 and extension post 64 can be inserted into stem channel 54. Furthermore, tibial stem 14 of FIG. 1 can be inserted into stem channel 54 after trialing and straight or offset stem provisional 62 and extension post 64 are removed. Tibial stem 14 can provide anchoring of tibial component 10 to tibia T.

Tibial component 10 can be further anchored to tibia T using sleeve 16 of FIG. 1. Reaming channel 52 can comprise a widened and tapered portion of stem channel 54 shaped to receive sleeve 16. Reaming head 58 can have the same outer angular dimensions as sleeve 16. That is, the angles of the side walls relative to the inferior and superior wall can be the same. As shown in FIG. 3, the shape of reaming channel 52 is typically symmetric to accommodate sleeve 16, which can have a similar shape. For example, the anterior-posterior thickness of sleeve 16 can be uniform in the central portion of the device. Additionally, the slope on the anterior and posterior walls can be the same. Sleeves or cones can additionally have non-uniform thicknesses or differently sloped sidewall can be used.

FIG. 3 is a side cross-sectional view of the proximal end of tibia T of FIG. 2 with reaming tool 50 removed and epiphysis end E of tibia T resected at resected surface 70. Reaming channel 52 can include tapered portion 72 and longitudinal portion 74. Longitudinal portion 74 can have length L1, which can be measured from resected surface 70. In other words, tapered portion 72 can begin a distance equal to length L1 below resected surface 70. Tapered portion 72 can have a longitudinal length L2 equivalent to the height of reaming head 58. Additionally, the angle between longitudinal portion 74 and tapered portion 72 can match with the geometer of reaming head 58. After reaming with reaming head 58, epiphysis E is resected to provide a planar, or nearly planar, surface for engaging flush with tibial tray 12 (FIG. 1) at resected surface 70. Additionally, re-sectioning of tibia T can be performed prior to reaming. Note, longitudinal portion 74 typically results from reaming tool 50 being advanced in a straight superior-inferior direction. Longitudinal portion 74 can be eliminated, partially or fully, by the introduction of pivoting and articulating between stem extension post 60 and stem provisional 62.

FIG. 4 is a side cross-sectional view of tibial component 10 and sleeve 16 of FIG. 1 inserted into reamed intramedullary canal C of FIG. 3. In the configuration of FIG. 4, sleeve 16 is attached to stem housing 24. Sleeve 16 can be attached to stem housing 24 in a variety of configurations, such as via threaded engagement, ribbed coupling (e.g., where shallow ribs on stem housing 24 engage with shallow ribs on sleeve 16), snap fit, force fit, press fit, Morse taper, or via use of additional fasteners. In the illustrated embodiment, sleeve 16 is attached to stem housing 24 via Morse taper. In examples, outer surface 44 of stem housing 24 is configured to have a Morse taper and interior channel 38 of sleeve 16 is configured to have a mating recess such that a self-holding connection is made. Such a configuration is discussed in greater detail in U.S. Pat. No. 6,911,100 to Gibbs et al., which is hereby incorporated by reference in its entirety for all purposes. In other examples, other tapered connections can be used, such as described in U.S. Pub. No. 2015/0216667 to Monaghan, which is hereby incorporated by reference in its entirety for all purposes. In yet other examples, sleeve 16 can be coupled to bone-facing surface 18 rather than stem housing 24.

With sleeve 16 connected to stem housing 24, sleeve 16 contacts tibia T at tapered portion 72 of reaming channel 52. Longitudinal portion 74 is small to permit exterior surface 32 to engage tapered portion 72 while still allowing gap G1 to be present between bone-facing surface 18 of tibial tray 12 and proximal portion 34 of sleeve 16. Gap G1 can be filled with bone cement. For example, gap G1 and reaming channel 52 can be filled with bone cement prior to insertion of tibial stem 14 into reaming channel 52. This can permit gap G2 along distal portion 36 to fill with bone cement.

Sleeve 16 can be attached to stem housing 24 in a coupled configuration as discussed. Sleeve 16 can additionally be inserted into reamed intramedullary canal C of FIG. 3 in an un-coupled configuration. As such, sleeve 16 can be not attached to stem housing 24. In such a configuration (e.g., unattached to stem housing 24), sleeve 16 can be referred to as a cone.

In either case, it can be desirable to have exterior surface 32 closely conform to walls of a bone pocket reamed or otherwise formed into a bone in order to, among other things, facilitate bone growth into sleeve 16, while the inner surface is brought toward outer surface 44 of stem housing 24. With the present disclosure, the space between interior channel 38 and exterior surface 32 of sleeve 16 can be used to store or house other components or materials that can enhance the functionality of sleeve 16. FIGS. 1-4 are discussed with reference to sleeves used with a tibial implant. However, the systems, devices and methods of the present disclosure can be used with cones or other implantable anchoring devices and in other bones, particularly other long bones, such as femurs.

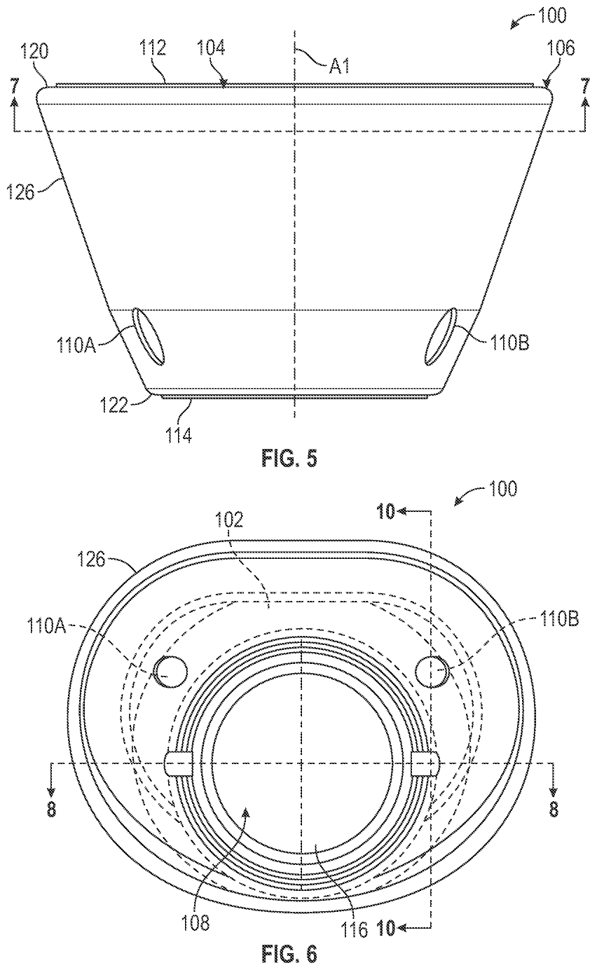

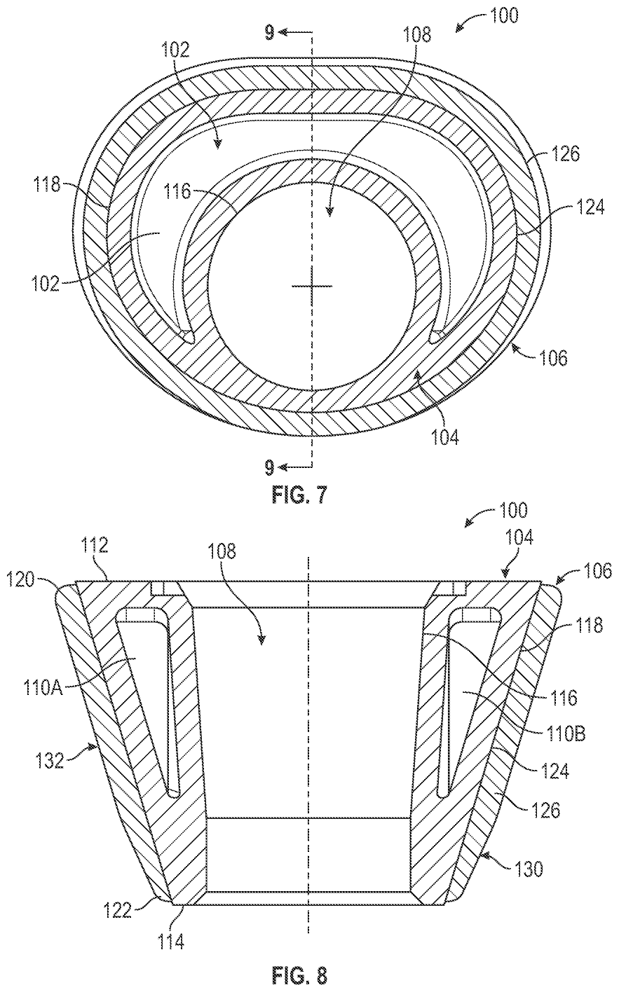

FIG. 5 is a front view of sleeve 100 having internal void 102 (FIGS. 6-7) within inner solid component 104, which is surrounded by outer porous component 106. FIG. 6 is a top view of sleeve 100 of FIG. 5 showing stem socket 108 and internal void 102 connected to passage 110A and passage 110B in phantom. FIG. 7 is cross-sectional view of sleeve 100 of FIG. 5 taken at section 7-7 to show internal void 102 extending along a posterior side of stem socket 108. FIGS. 5-7 are discussed concurrently.

Inner solid component 104 can have upper surface 112, lower surface 114, inner surface 116 and outer surface 118. In examples, inner solid component 104 can comprise a monolithic or single-piece unit of material. For example, inner solid component 104 can comprise a block of stainless steel or titanium that can be produced by a casting process or an additive manufacturing process. The material of inner solid component 104 can be solid so as to not include any micro-pores or crevices. The exposed surfaces of inner solid component 104, such as inner surface 116, can be smooth to engage flush with other components, such as a tibial stem.

Outer porous component 106 can comprise upper surface 120, lower surface 122, inner surface 124 and outer surface 126. In examples, outer porous component 106 can comprise a monolithic or single-piece unit of material. For example, outer porous component 106 can comprise a webbed or ligamentous structure of material, such as porous tantalum fabricated from a vapor deposition process (e.g. Trabecular Metal™, commercially available from Zimmer Biomet) or porous Ti6AI4V alloy material fabricated from a three-dimensional printing process (e.g. OsseioTi®, commercially available from Zimmer Biomet). The material of outer porous component 106 can be porous (e.g., include micro-pores) so as to include voids or open spaces to facilitate elution of treatment fluid. The exterior surfaces of outer porous component 106 can be rough to allow for tissue ingrowth.

Outer porous component 106 can be attached or affixed to inner solid component 104. For example, inner surface 124 of outer porous component 106 can abut outer surface 118 of inner solid component 104. As such, inner surface 124 can be bonded or joined to outer surface 118 via an appropriate process, such as welding or fusion bonding. In examples, outer porous component 106 can be produced directly onto outer surface 118 via a vapor deposition process or an additive manufacturing process. In additional examples, inner solid component 104 and outer porous component 106 can be fabricated simultaneously using a single manufacturing process, such as a vapor deposition process or an additive manufacturing process.

In the illustrated example, internal void 102 can be positioned within inner solid component 104 so as to be completely bounded by the material of inner solid component 104, with the exception of passage 110A and passage 110B. Internal void 102 can be disposed between outer surface 118 and inner surface 116. Passage 110A and passage 110B can extend through outer porous component 106 and into inner solid component 104 to intersect internal void 102. Specifically, passage 110A and passage 110B can extend into outer surface 126, through inner surface 124 and into outer surface 118.

Passage 110A and passage 110B can be used to facilitate manufacture of internal void 102 and provide functionality to internal void 102. For example, internal void 102 can be produced using an additive manufacturing process, such as select laser metal sintering wherein layers of powdered metal material are melted using a laser beam where the component is intended to occupy space while the powder is left loose where the component is not intended to occupy space. Thus, the laser can engage powder in places between inner surface 116 and outer surface 118 and can avoid powder in places where internal void 102 is to be present. The non-sintered powdered metal is removed from the final component. Passage 110A and 110B can be produced in sleeve 100 using machining processes after sleeve 100 is fabricated, or can be produced with sleeve 100, such as during an additive manufacturing process. Passage 110A and passage 110B can be used to facilitate removal of metal powder from internal void 102. The presence of internal void 102, passage 110A and passage 110B can reduce the amount of material of sleeve 100, thereby reducing the weight of sleeve 100. A reduction in the weight of sleeve 100 can produce a better experience for a patient in which sleeve 100 is implanted. Furthermore, powdered metal recovered from internal void 102 can be collected and reused in other additive manufacturing processes.

Passage 110A and passage 110B can additionally be used to facilitate placement of a material into internal void 102 that can provide functionality to sleeve 100. In examples, internal void 102 can be filled, partially or completely, with various substances that have clinical or biological effects. In examples, internal void 102 can be provided with treatment fluids as described herein. In examples, internal void 102 can be provide with other types of materials, including fluids, solids and pastes that can have various biological effects, treatment effects and clinical effects.

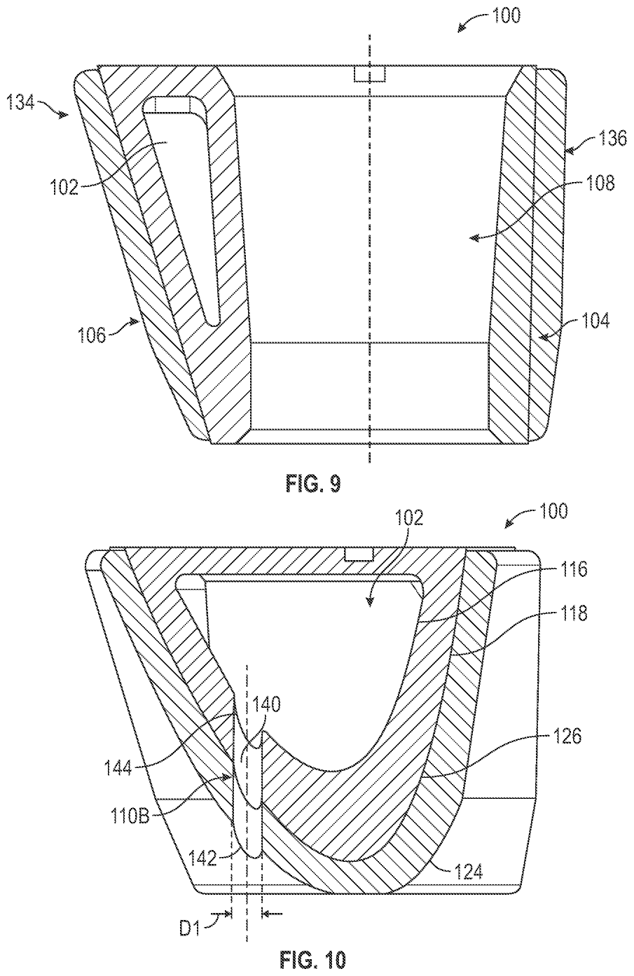

FIG. 8 is cross-sectional view of sleeve 100 of FIG. 6 taken at section 8-8 to show internal void 102 positioned on medial and lateral sides of stem socket 108. FIG. 9 is cross-sectional view of sleeve 100 of FIG. 7 taken at section 9-9 to show internal void 102 positioned on a posterior side of stem socket 108. FIG. 10 is cross-sectional view of sleeve 100 of FIG. 6 taken at section 10-10 to show passage 110B extending from outer surface 126 of sleeve 100 to internal void 102. FIGS. 8-10 are discussed concurrently.

As discussed above, typical cones and sleeves are fabricated to have conical or conical-like bodies that have uniform or nearly-uniform thicknesses between the exterior surface and the stem socket. Internal voids of the present disclosure can be placed in such walls. However, the wall thicknesses in conical and conical-like bodies can be thin compared to what is desirable or advantageous for the placement of treatment fluids or electronics. With the present disclosure, sleeve 100 can have asymmetric shapes that produce sleeves having thick walls between stem socket 108 and one or more surfaces of outer surface 118, e.g., between inner surface 116 and outer surface 118. The thickness of such walls can be suitable for placement of treatment fluid or electronics.

As can be seen in FIG. 8, sleeve 100 can comprise first side portion 130 and second side portion 132. First side portion 130 and second side portion 132 can comprise medial and lateral sides of sleeve 100 depending on which of the left and right leg sleeve 100 is implanted. Stem socket 108 can extend along first axis A1, which can generally comprise the center of sleeve 100 in a transverse plane. At outer surface 126 and outer surface 118, first side portion 130 and second side portion 132 can be obliquely angled relative to first axis A1. Inner surface 116 of inner solid component 104 can be tapered to provide coupling to a tibial stem. Specifically, a bottom portion of inner surface 116 can be parallel to first axis A1, with an upper portion being tapered away from first axis A1 extending from the bottom portion. In examples, inner surface 116 can include a Morse taper. The angling of outer surface 118 away from first axis A1 can be greater than the tapering of inner surface 116 away from axis A1 such that first side portion 130 and second side portion 132 can increase in width from lower surface 114 to upper surface 112. The angling of outer surface 118 away from first axis A1 can provide space for internal void 102.

Internal void 102 can extend around stem socket 108 to the medial and lateral sides of stem socket 108. The width of internal void 102 between inner surface 116 and outer surface 118 can increase from proximate lower surface 114 to upper surface 112.

As can be seen in FIG. 9, sleeve 100 can comprise anterior wall 134 and posterior wall 136. Posterior wall 136 can be approximately parallel to first axis A1 of stem socket 108. Anterior wall 134 can be obliquely angled relative to first axis A1. Inner surface 116 of inner solid component 104 can be tapered to provide coupling to a tibial stem.

Within posterior wall 136, inner solid component 104 can be contiguous between inner surface 116 and outer surface 118, and outer porous component 106 can be contiguous between inner surface 124 and outer surface 126.

Within anterior wall 134, inner solid component 104 can be hollow between inner surface 116 and outer surface 118, and outer porous component 106 can be contiguous between inner surface 124 and outer surface 126.

As can be seen in FIG. 10, passage 110B can extend from internal void 102 to the exterior of sleeve 100. Specifically, passage 110B can extend into outer surface 126, through inner surface 124 and into outer surface 118 to reach internal void 102. Passage 110B can include sidewall 140 extending between inlet 142 and outlet 144. Passage 110A can be constructed similarly as passage 110B.

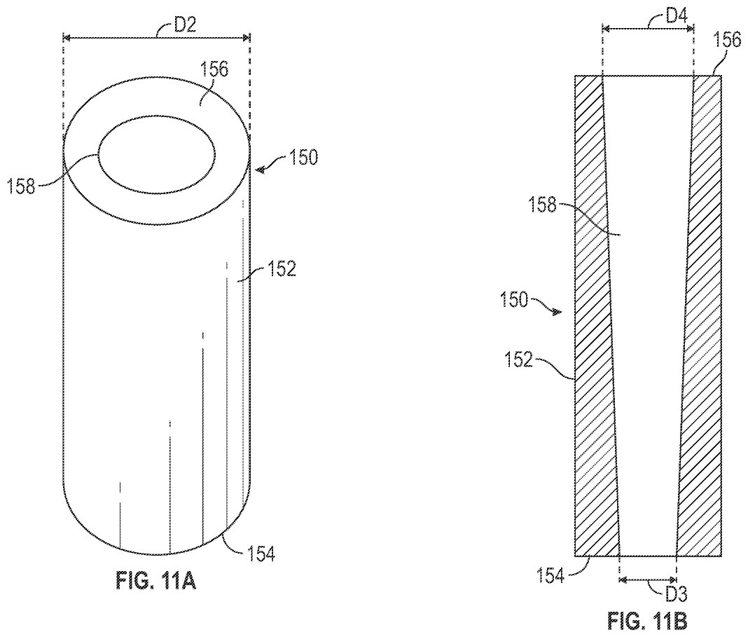

Passage 110A and passage 110B can be sized for different purposes, such as to facilitate removal of metal powder from internal void 102, to facilitate filling of internal void 102 with treatment fluid, and to facilitate eluting of treatment fluid from internal void 102. Passages 110A and 110B can each have diameter D1. It can be desirable for diameter D1 to be sufficiently large to allow metal powder not consumed in additive manufacturing processes to be removed from internal void 102. For example, in order to ensure that sleeve 100 is sufficiently clean to be implanted within anatomy and filled with treatment fluid, it can be desirable to ensure that internal void 102 is cleared of loose debris and the like. Additionally, it can be desirable for diameter D1 to be relatively large to allow for treatment fluid to be added to internal void 102, such as via an injection process or the like. However, in order to prevent treatment fluid from exiting internal void faster than is desired for appropriate eluting of treatment fluid, it can be desirable for diameter D1 to be relatively smaller compared to diameter sizes suitable for removing debris and powder. In examples, passage 110A and passage 110B can receive plug 150 (FIGS. 11A and 11B) that can be used to control egress and ingress of matter from internal void 102.

FIG. 11A is a perspective view of plug 150 that can be inserted into passage 110A or passage 110B of FIGS. 5, 6 and 10. FIG. 11B is a cross-sectional side view of plug 150 of FIG. 11A. Plug 150 can comprise an elongate body having sidewall 152, first end 154 and second end 156. Aperture 158 can extend from first end 154 to second end 156. FIG. 11A and FIG. 11B are discussed concurrently.

Plug 150 can be configured to be positioned within passage 110A or passage 110B to limit or inhibit egress of treatment fluid from internal void 102. Plug 150 can have outer diameter D2. In examples, diameter D2 can be sized to produce an interference fit (e.g., press fit or force fit) with passage 110A and passage 110B. In examples, plug 150 can include exterior (e.g., male) threading configured to engage mating interior (e.g., female) threading in passage 110A and passage 110B. As such diameter D2 can be approximately equal to, or slightly larger than diameter D1. Aperture 158 can have diameter D3 at first end 154 and diameter D4 at second end 156. In examples, aperture 158 can comprise a metering passage where D3 and D4 are different, as illustrated. In other examples, D3 and D4 can be equal such that aperture 158 is a straight through-bore.

Sleeve 100 can be provided with a pair of plugs 150 for passage 110A and passage 110B. Sleeve 100 can include a plug for each of the passages within sleeve 100. In other examples, sleeve 100 can include fewer plugs than there are passages within sleeve 100.

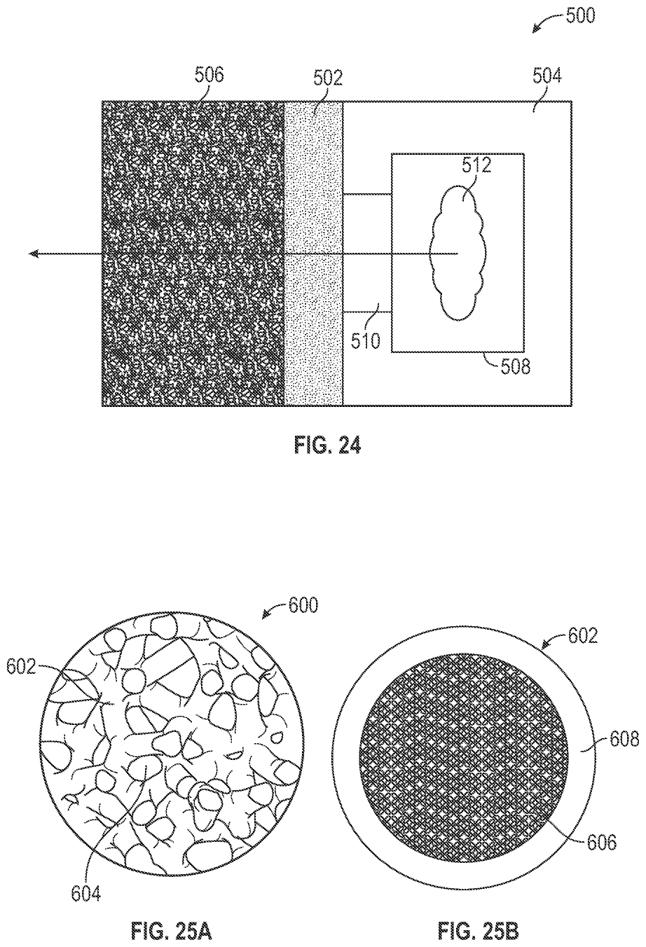

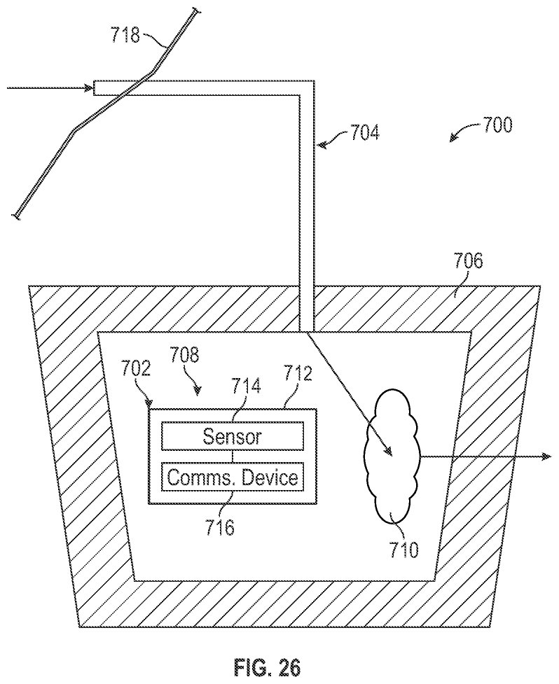

After sleeve 100 has been fabricated and passage 110A and passage 110B have been produced, any un-sintered (e.g., unmelted) metal powder or other debris within internal void 102 can be removed, such as via one or both of suctioning and washing. Sleeve 100 can be washed, sanitized and sterilized as appropriate. Internal void 102 can be filled with treatment fluid as described herein using any suitable process, such as injection, pouring, troweling and the like. Internal void 102 can be filled pre-operatively, such as at a manufacturing facility or can be filled intra-operatively. Use of membranes as described with reference to FIG. 24 with sleeve 100 can facilitate retention of treatment fluid within internal void 102. Additionally, internal void 102 can be filled post-operatively using a tubing system, as described with reference to FIG. 26. For pre- and intra-operative filling, after the desired amount of treatment fluid is positioned within internal void 102, an instance of plug 150 can be inserted into one or both of passage 110A and passage 110B. Plug 150 can be inserted into passage 110A or passage 110B with either first end 154 or second end 156 being positioned within sleeve 100 to produce desired metering effects. One or both of first end 154 and second end 156 can be shaped or contoured to match with the curvature of outer surface 126. In examples, plug 150 can have a length less than the lengths of passage 110A and passage 110B such that plug 150 can be fully recessed within passage 110A or passage 110B.

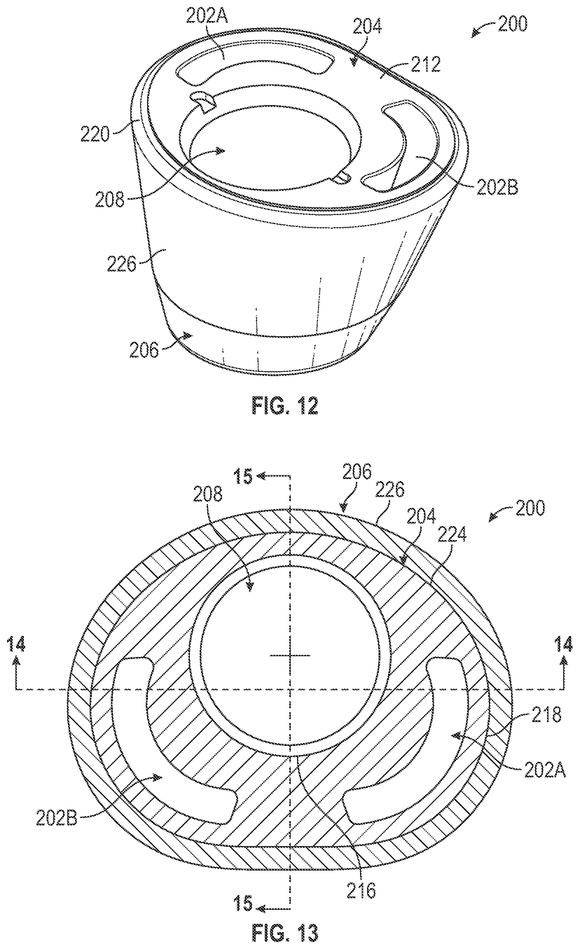

Configured as described with reference to FIGS. 5-11B, sleeve 100 can include supplemental functionality in addition to bone-anchoring functionality. For example, internal void 102 can include treatment fluids or electronics that can be placed in space within sleeve 100 that would typically be occupied by material of the sleeve. FIGS. 5-10 describe an example of internal void 102 fully located within inner solid component 104. However, the space between stem socket 108 and outer surface 126 can be utilized in other ways without affecting the outer shape of sleeve 100, as is described with reference to FIGS. 12-26.

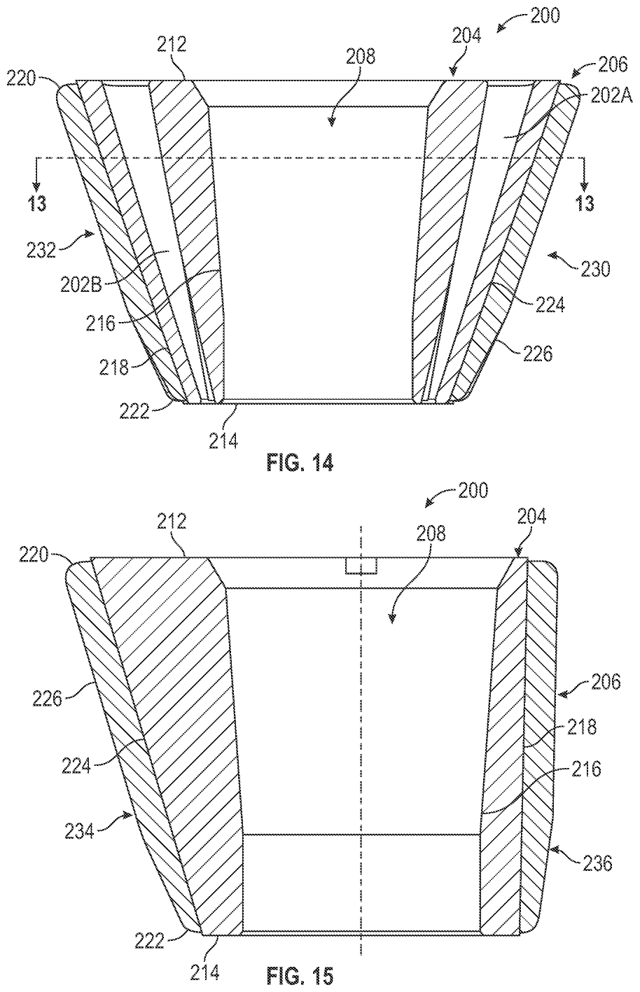

FIG. 12 is a perspective view of sleeve 200 having internal void 202A and internal void 202B extending through inner solid component 204, which can be surrounded by outer porous component 206. FIG. 13 is a top cross-sectional view of sleeve 200 of FIG. 12 showing internal void 202A and internal void 202B extending within material of inner solid component 204. Stem socket 208 can be located in inner solid component 204. FIG. 14 is a front cross-sectional view of sleeve 200 of FIG. 12 showing internal void 202A and internal void 202B extending from upper surface 212 to lower surface 214. FIG. 15 is a side cross-sectional view of sleeve 200 of FIG. 15 showing an interface between inner solid component 204 and outer porous component 206 at anterior and posterior sides of sleeve 200. FIGS. 12-15 are discussed concurrently.

Inner solid component 204 can have upper surface 212, lower surface 214, inner surface 216 and outer surface 218. In examples, inner solid component 204 can comprise a monolithic or single-piece unit of material. For example, inner solid component 204 can comprise a block of stainless steel or titanium that can be produced by a casting process or an additive manufacturing process. The material of inner solid component 204 can be solid so as to not include any micro-pores or crevices. The exposed surfaces of inner solid component 204, such as inner surface 216, can be smooth to engage flush with other components, such as a tibial stem.

Outer porous component 206 can comprise upper surface 220, lower surface 222, inner surface 224 and outer surface 226. In examples, outer porous component 206 can comprise a monolithic or single-piece unit of material. For example, outer porous component 206 can comprise a webbed or ligamentous structure of material. The material of outer porous component 206 can be porous (e.g., include micro-pores) so as to include voids or open spaces to facilitate elution of treatment fluid. The exterior surfaces of outer porous component 206 can be rough to allow for tissue ingrowth.

Sleeve 200 can comprise first side portion 130, second side portion 132, anterior wall 234 and posterior wall 236. First side portion 230 and second side portion 232 can comprise medial and lateral sides of sleeve 200 depending on which of the left and right leg sleeve 200 is implanted.

Sleeve 200 can be configured similarly as sleeve 100 of FIGS. 5-10 with differences including that there are two internal voids (i.e., internal void 202A and internal void 202B) rather than one internal void (i.e., internal void 102) and each of internal void 202A and internal void 202B extends through to upper surface 212 and lower surface 214 of inner solid component 204 rather than internal void 102 being completely bounded by material of inner solid component 104. Internal void 202A and internal void 202B can provide direct access to the exterior of sleeve 200 for emptying debris and powdered metal, filling with treatment fluid and allowing treatment fluid to elute. For example, internal void 202A and internal void 202B can have cross-sectional areas, as viewed in FIG. 13, that facilitate placement of treatment fluid into internal void 202A and internal void 202B.

As can be seen in FIG. 14, the cross-sectional area of internal void 202A and internal void 202B can decrease from a superior portion near upper surface 212 to an inferior portion near lower surface 214. Such a shape can facilitate filling of internal void 202A and internal void 202B near upper surface 212, while allowing eluting of treatment fluid near lower surface 214.

In examples, internal void 202A and internal void 202B can extend all the way from upper surface 212 to lower surface 214. In examples, internal void 202A and internal void 202B can be configured to not penetrate through one of upper surface 212 and lower surface 214. For example, the upper portions of internal void 202A and internal void 202B can be open to allow for filling, while the lower portions of internal void 202A and internal void 202B near lower surface 214 can be closed.

Sleeve 200 is illustrated as not having any passages through outer porous component 206 that reach internal void 202A and internal void 202B. However, in examples, sleeve 200 can be provided with one or more passages that penetrate into outer surface 226 and extend into either internal void 202A and internal void 202B.

Sleeve 200 is illustrated as having two separate internal voids. Such a configuration can be useful in providing a first internal void for placement of treatment fluid and a second internal void for placement of an electronics module, as shown in FIG. 26. For example, internal void 202A can be filled with treatment fluid and can include a passage to outer surface 226, while internal void 202B can include an electronics module and not include a passage to outer surface 226. However, in additional examples, internal void 202A and internal void 202B can be connected along anterior wall 234 to provide a single internal void.

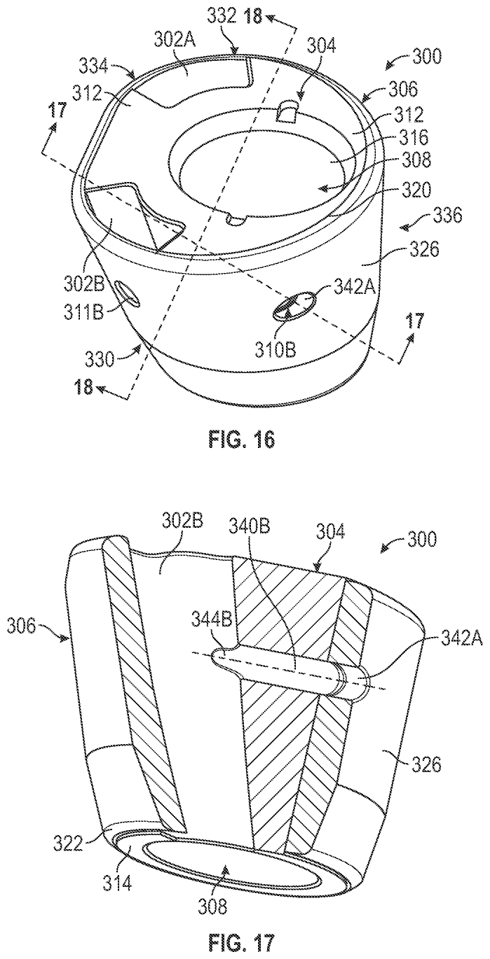

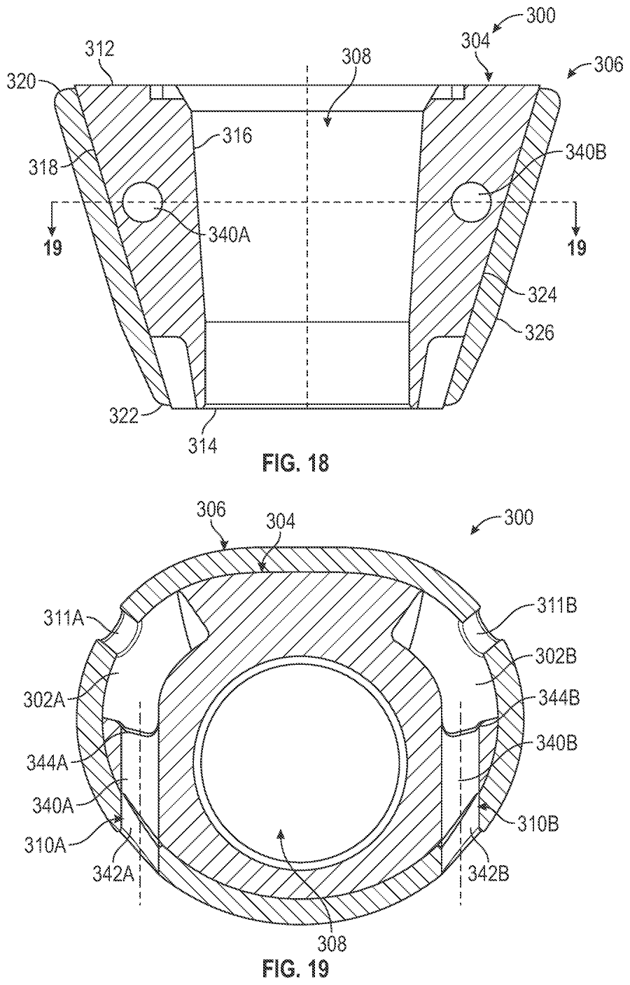

FIG. 16 is a perspective view of sleeve 300 having internal void 302A and internal void 302B between inner solid component 304 and outer porous component 306. Stem socket 308 can be located in inner solid component 304. FIG. 17 is a top view of sleeve 300 of FIG. 16 showing internal void 302A and internal void 302B connected to passage 310A and passage 310B, respectively, and orifice 311A and 311B, respectively. FIG. 18 is a front cross-sectional view of sleeve 300 of FIG. 17 taken at section 18-18 to show passage 310A and passage 310B in inner solid component 304. FIG. 19 is a top cross-sectional view of sleeve 300 of FIG. 18 to show passage 310A and passage 310B extending between inner solid component 304 and outer porous component 306. FIGS. 16-19 are discussed concurrently.

Inner solid component 304 can have upper surface 312, lower surface 314, inner surface 316 and outer surface 318. In examples, inner solid component 304 can comprise a monolithic or single-piece unit of material. For example, inner solid component 304 can comprise a block of stainless steel or titanium that can be produced by a casting process or an additive manufacturing process. The material of inner solid component 304 can be solid so as to not include any micro-pores or crevices. The exposed surfaces of inner solid component 304, such as inner surface 316, can be smooth to engage flush with other components, such as a tibial stem.

Outer porous component 306 can comprise upper surface 320, lower surface 322, inner surface 324 and outer surface 326. In examples, outer porous component 306 can comprise a monolithic or single-piece unit of material. For example, outer porous component 306 can comprise a webbed or ligamentous structure of material. The material of outer porous component 306 can be porous (e.g., include micro-pores) so as to include voids or open spaces to facilitate elution of treatment fluid. The exterior surfaces of outer porous component 306 can be rough to allow for tissue ingrowth.

Sleeve 300 can comprise first side portion 330, second side portion 332, anterior wall 334 and posterior wall 336. First side portion 330 and second side portion 332 can comprise medial and lateral sides of sleeve 300 depending on which of the left and right leg sleeve 300 is implanted.

Passage 310A can include sidewall 340A extending between inlet 342A and outlet 344A. Passage 310B can include sidewall 340B extending between inlet 342B and outlet 344B.

Sleeve 300 can be configured similarly as sleeve 200 of FIGS. 11-14 with differences including that internal void 302A and internal void 302B penetrating through material of outer surface 318 of inner solid component 304 rather than internal void 202A and internal void 202B being bounded by material of outer surface 218. Additionally, passage 310A and passage 310B can fluidly connect internal void 302A and internal void 302B to posterior wall 336 of sleeve 300. Internal void 302A and internal void 302B can provide direct access to the exterior of sleeve 300 for emptying debris and powdered metal, filling with treatment fluid and allowing treatment fluid to elute. For example, internal void 302A and internal void 302B can have cross-sectional areas, as viewed in FIG. 19, that facilitate placement of treatment fluid into internal void 302A and internal void 302B. Furthermore, internal void 302A and internal void 302B provide direct access to outer porous component 306 to allow for treatment fluid to elute through the pores of outer porous component 306.

As can be seen in FIG. 17, the cross-sectional area of internal void 302B can decrease from a superior portion near upper surface 312 to an inferior portion near lower surface 314. Such a shape can facilitate filling of internal void 302B near upper surface 312, while allowing eluting of treatment fluid near lower surface 314. Internal void 302A can be configured similarly as internal void 302B. As compared to internal void 202A and internal void 202B of FIGS. 12-16, internal void 302A and internal void 302B can be wider due to such internal voids extending all the way to outer porous component 306. As such, the volumes of internal void 302A and internal void 302B can be larger than the volumes of internal void 202A and internal void 202B. However, inner solid component 304 can still provide support to outer porous component 306 along the length of inner solid component 304 between upper surface 32 and lower surface 314 in places between internal void 302A and internal void 302B.

In examples, internal void 302A and internal void 302B can extend all the way from upper surface 312 to lower surface 314. In examples, internal void 302A and internal void 302B can be configured to not penetrate through one of upper surface 312 and lower surface 314. For example, the upper portions of internal void 302A and internal void 302B can be open to allow for filling, while the lower portions of internal void 302A and internal void 302B near lower surface 314 can be closed.

Sleeve 300 is illustrated as having two passages, e.g., passage 310A and passage 310B, through outer porous component 306 and portions of inner solid component 304 that reach internal void 302A and internal void 302B. Additionally, sleeve 300 is illustrated as having two orifices, e.g., orifice 311A and orifice 311B, extending through outer porous component 306 to directly reach internal void 302A and internal void 302B. In examples, only one of passage 310A and passage 310B and only one of orifice 311A and orifice 311B can be included. The diameters of passages 310A and 310B can be different than the diameters of orifice 311A and orifice 311B. For example, the diameter of orifice 311A and orifice 311B can be relatively large to allow for filling and emptying of internal void 302A and internal void 302B, while the diameters of passage 310A and 310B can be relatively small to provide for elution of treatment fluid, or vice versa. Any combination of passage 310A, passage 310B, orifice 311A and orifice 311B can be provided with a plug, such as plug 150 of FIGS. 11A and 11B or a solid plug (e.g., without aperture 158), to prevent or limit elution of treatment fluid.

Sleeve 300 is illustrated as having two separate internal voids. Such a configuration can be useful in providing a first internal void for placement of treatment fluid and a second internal void for placement of an electronics module, as shown in FIG. 26. For example, internal void 302A can be filled with treatment fluid and can include a passage to outer surface 326, while internal void 302B can include an electronics module and not include a passage to outer surface 326. However, in additional examples, internal void 302A and internal void 302B can be connected along anterior wall 334 to provide a single internal void.

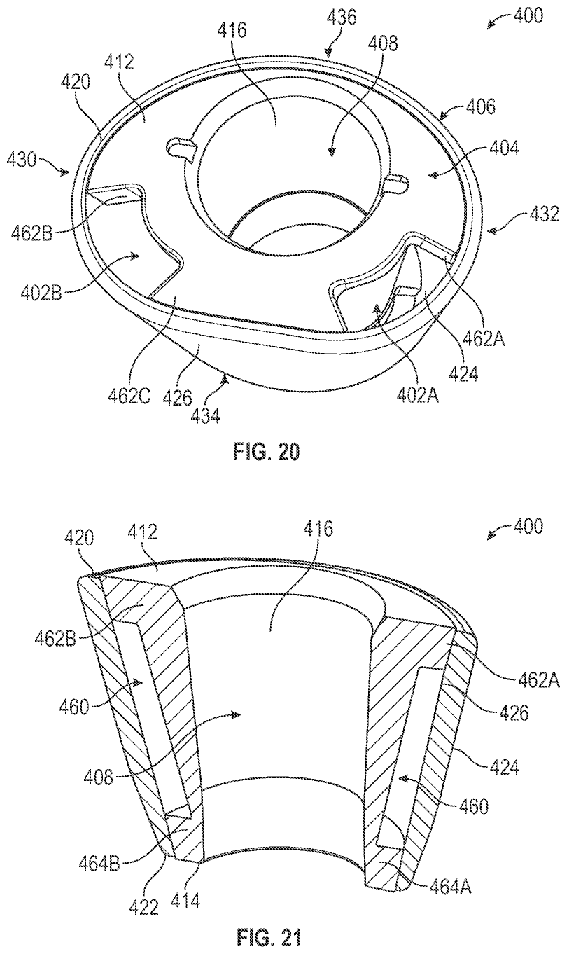

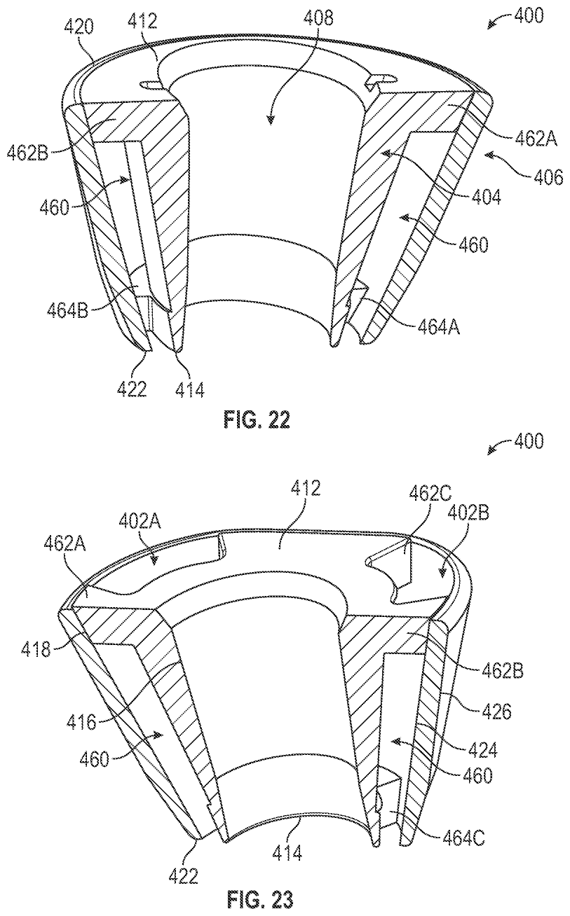

FIG. 20 is a perspective view of sleeve 400 having internal void 402A and internal void 402B between inner solid component 404 and outer porous component 406 that are connected by central void 460. Stem socket 408 can be located in inner solid component 404. FIG. 21 is a cross-sectional view of sleeve 400 of FIG. 20 showing upper supports 462A and 462B and lower supports 464A and 464B on inner solid component 404 for outer porous component 406. FIG. 22 is a cross-sectional view of sleeve 400 of FIG. 20 showing internal void 402A and internal void 402B extending to lower surface 414 of sleeve 400. FIG. 23 is a cross-sectional view of sleeve 400 of FIG. 20 showing central void 460 in fluid communication with internal void 402A and internal void 402B, upper support 462C and lower support 464C. FIGS. 20-23 are discussed concurrently.

Inner solid component 404 can have upper surface 412, lower surface 414, inner surface 416 and outer surface 418. In examples, inner solid component 404 can comprise a monolithic or single-piece unit of material. For example, inner solid component 404 can comprise a block of stainless steel or titanium that can be produced by a casting process or an additive manufacturing process. The material of inner solid component 404 can be solid so as to not include any micro-pores or crevices. The exposed surfaces of inner solid component 404, such as inner surface 416, can be smooth to engage flush with other components, such as a tibial stem.

Outer porous component 306 can comprise upper surface 420, lower surface 422, inner surface 424 and outer surface 426. In examples, outer porous component 406 can comprise a monolithic or single-piece unit of material. For example, outer porous component 406 can comprise a webbed or ligamentous structure of material. The material of outer porous component 106 can be porous (e.g., include micro-pores) so as to include voids or open spaces to facilitate elution of treatment fluid. The exterior surfaces of outer porous component 406 can be rough to allow for tissue ingrowth.

Sleeve 400 can comprise first side portion 430, second side portion 432, anterior wall 434 and posterior wall 436. First side portion 430 and second side portion 432 can comprise medial and lateral sides of sleeve 400 depending on which of the left and right leg sleeve 400 is implanted.

Sleeve 400 can be configured similarly as sleeve 300 of FIGS. 16-19 with differences including that internal void 402A and internal void 402B can be connected by central void 460 and passage 310A and passage 310B can be omitted. Central void 460 can comprise an annular void at least partially circumscribing, e.g., extending at least partially around, stem socket 408 similar to internal void 102 as can be seen in FIG. 7. As such, central void 460 can have a C-shape or crescent shape cross-section. Thus, the internal void space of sleeve 400 can be a combination of the internal void spaces of sleeve 100 and sleeve 300.

Internal void 402A and internal void 402B can provide direct access to the exterior of sleeve 400 for emptying debris and powdered metal, filling with treatment fluid and allowing treatment fluid to elute. For example, internal void 402A and internal void 402B can have cross-sectional areas, as viewed in FIG. 20, that facilitate placement of treatment fluid into internal void 402A and internal void 402B. Furthermore, internal void 402A and internal void 402B provide direct access to outer porous component 406 to allow for treatment fluid to elute through the pores of outer porous component 406.

The cross-sectional area of internal void 402B can decrease from a superior portion near upper surface 412 to an inferior portion near lower surface 414, similar to what is shown with reference to sleeve 300 of FIG. 17. Such a shape can facilitate filling of internal void 402B near upper surface 412, while allowing eluting of treatment fluid near lower surface 414. Internal void 402A can be configured similarly as internal void 402B.

In examples, internal void 402A and internal void 402B can extend all the way from upper surface 412 to lower surface 414. In examples, internal void 402A and internal void 402B can be configured to not penetrate through one of upper surface 412 and lower surface 414. For example, the upper portions of internal void 402A and internal void 402B can be open to allow for filling, while the lower portions of internal void 402A and internal void 402B near lower surface 414 can be closed.

Sleeve 400 is illustrated as not having any passages, such as passage 310A and passage 310B or orifices, such as orifice 311A and orifice 311B, of FIG. 19. However, sleeve 400 can be provided with similar passages and orifices, as described with reference to FIG. 19.

Internal void 402A and internal void 402B can be connected by central void 460 to produce a large chamber within sleeve 400, as compared to other sleeves described herein. Central void 460 can increase the storage volume within sleeve 400 for the presence of treatment fluid and electronics. As such, sleeve 400 can take advantage of a large percentage of the space between inner surface 416 and outer surface 418. Upper supports 462A, 462B and 462C and lower supports 464A, 464B and 464C can provide structural support of inner solid component 404 against outer porous component 406. Upper supports 462A, 462B and 462C and lower supports 464A, 464B and 464C can additionally provide surface area for bonding between inner solid component 404 and outer porous component 406.

FIG. 24 is a schematic diagram of sleeve 500 comprising membrane 502 positioned between inner solid component 504 and outer porous component 506. Inner solid component 504 can comprise internal void 508 having passage 510 extending therefrom to the exterior of inner solid component 504. Treatment fluid 512 can be placed within internal void 508. Membrane 502 can be positioned alongside inner solid component 504 to cover passage 510. Passage 510 may be tubular or cuboidal with predefined dimensions. Membrane 502 can be used to control the release or elution of treatment fluid 512 from internal void 508.

Inner solid component 504 can comprise any of the inner solid components described herein, such as inner solid component 104 (FIG. 5), inner solid component 204 (FIG. 12), inner solid component 304 (FIG. 16) and inner solid component 404 (FIG. 20).

Outer porous component 506 can comprise any of the outer porous components described herein, such as outer porous component 106 (FIG. 5), outer porous component 206 (FIG. 12), outer porous component 306 (FIG. 16) and outer porous component 406 (FIG. 20).

Membrane 502 can be configured to keep treatment fluid 512 maintained within internal void 508 until sleeve 500 has been implanted within anatomy.

In examples, membrane 502 can comprise an impermeable barrier to prevent treatment fluid 512 from passing therethrough until membrane 502 is broken down by biological fluid of the patient, thereafter allowing release of treatment fluid 512 into the anatomy. Examples of impermeable barriers comprise gelatin (hard or soft) and non-gelatin shells generally derived from hydrolysis of collagen (acid, alkaline, enzymatic, or thermal hydrolysis) from animal origin or cellulose based.

In examples, membrane 502 can comprise a permeable barrier to temporarily prevent treatment fluid 512 from passing therethrough until a certain amount of time elapses, thereafter allowing release of treatment fluid 512 into the anatomy. Examples of permeable barriers comprise cellulose acetate, Nitrocellulose, and cellulose esters (CA, CN, and CE), polysulfone (PS), polyether sulfone (PES), polyacrilonitrile (PAN), polyamide, polyimide, polyethylene and polypropylene (PE and PP), polytetrafluoroethylene (PTFE), polyvinylidene fluoride (PVDF), polyvinylchloride (PVC).

Gravity and movement of the patient into which sleeve 500 is implanted can facilitate migration or elution of treatment fluid 512 from internal void 508, through passage 510, through membrane 502, through outer porous component 506 and into the anatomy.

FIG. 25A is a close-up diagrammatic view of porous material 600 suitable for use with the present disclosure comprising a plurality of ligaments 602 forming open spaces 604. FIG. 25B is a schematic cross-sectional view of one of ligaments 602 of FIG. 25A showing carbon core 606 surrounded by film 608. FIGS. 25A and 25B are discussed concurrently.

Porous material can comprise ligaments 602 and open spaces 604. FIGS. 25A and 25B are not necessarily drawn to scale in order to more readily visualize the features of porous material 600. Additionally, the number of ligaments 602 is fewer than can be used in practice. Likewise, the density of ligaments 602 can be greater in practice. Furthermore, the density of ligaments 602 can be greater in practice.

Porous material 600 can be formed of a suitable material that promotes bone ingrowth and is biocompatible, such as porous metal, or a porous tantalum having a porosity as low as 55%, 65%, or 75% and as high as 80%, 85%, or 90%, for example. An example of a highly porous tantalum material is produced using Trabecular Metal™ Technology generally available from Zimmer Biomet, of Warsaw, Ind. Trabecular Meta™ is a trademark of Zimmer Biomet. Such a material may be formed from a reticulated vitreous carbon foam substrate which is infiltrated and coated with a biocompatible metal, such as tantalum, by a chemical vapor deposition (CVD) process in the manner disclosed in detail in U.S. Pat. No. 5,282,861 to Kaplan, the disclosure of which is expressly incorporated herein by reference in its entirety for all purposes. Specifically, ligaments 602 can be formed by via a chemical vapor deposition process used to bond outer porous component 506 to inner solid component 504. In addition to tantalum, other metals such as niobium, or alloys of tantalum and niobium with one another or with other metals may also be used.

FIG. 25A shows porous material 600 comprising an open cell tantalum structure that can be used as the material for any of the outer porous components described herein, such as outer porous component 106 (FIG. 5), outer porous component 206 (FIG. 12), outer porous component 306 (FIG. 16) and outer porous component 406 (FIG. 20). The open cell metal structures can be fabricated using the tantalum metal film and carbon substrate combination, with the film deposited by CVD, which mimics bone closely in having ligaments 602 interconnected to form open spaces 604. With the variables available in both the materials and the fabrication process, e.g., carbon core 606 and film 608, it is possible to obtain the simultaneous optimization of multiple properties (e.g. strength, stiffness, density, weight, the ability of treatment fluid to pass through) in ligaments 602 for the given application of substitution for bone. Although porous material 600 is shown as being porous in the form of an open cell tantalum structure, it is contemplated that in other examples porous structures can be used with the sleeve structures described herein.

Generally, the porous tantalum structure can include a large plurality of ligaments 602 defining open spaces 604 therebetween, with each ligament 602 generally including a carbon core 606 covered by film 608 of metal such as tantalum, for example. Open spaces 604 between ligaments 602 form a matrix of continuous channels having no dead ends, such that growth of cancellous bone through the porous tantalum structure, and passage of treatment fluid through the porous tantalum structure, is uninhibited. The porous tantalum may include up to 75%-85% or more void space therein. Thus, porous tantalum is a lightweight, strong porous structure which is substantially uniform and consistent in composition, and closely resembles the structure of natural cancellous bone, thereby providing a matrix into which cancellous bone may grow to provide fixation of the sleeves described herein to bone.

The porous tantalum structure may be made in a variety of densities in order to selectively tailor the structure for particular applications. In particular, as discussed in the above-incorporated U.S. Pat. No. 5,282,861, the porous tantalum may be fabricated to virtually any desired porosity and pore size, and can thus be matched with the surrounding natural bone in order to provide an improved matrix for bone ingrowth and mineralization. Furthermore, the density of the porous tantalum may be made to have varying densities within porous material 600 to provide various features to porous material 600.

Sleeves with porous structures can be provided by any number of suitable three-dimensional, porous structures, and these structures can be formed with one or more of a variety of materials including but not limited to polymeric materials which are subsequently pyrolyzed, metals, metal alloys, ceramics. In some instances, a highly porous three-dimensional structure can be fabricated using a selective laser sintering (SLS) process, or other additive manufacturing-type and three-dimensional printing type processes, such as direct metal laser sintering. In one example, a three-dimensional porous article is produced in layer-wise fashion from a laser-fusible powder, e.g., a polymeric material powder or a single-component metal powder, that is deposited one layer at a time. The powder is fused, remelted or sintered, by the application of laser energy that is directed to portions of the powder layer corresponding to a cross section of the article. After the fusing of the powder in each layer, an additional layer of powder is deposited, and a further fusing step is carried out, with fused portions or lateral layers fusing so as to fuse portions of previous laid layers until a three-dimensional article is complete. In certain embodiments, a laser selectively fuses powdered material by scanning cross-sections generated from a 3-D digital description of the article, e.g., from a CAD file or scan data, on the surface of a powder bed. Net shape and near net shape constructs are infiltrated and coated in some instances. Specifically, in various embodiments, porous material 600 can be fabricated from material compatible with various rapid manufacturing processes. For example, porous material 600 can be fabricated from lasered metallic powder.

Complex geometries can be created using such techniques, including geometries of internal voids within structures described herein. In some instances, a three-dimensional porous structure will be particularly suited for contacting bone and/or soft tissue, and in this regard, can be useful as a bone substitute and as cell and tissue receptive material, for example, by allowing tissue to grow into the porous structure over time to enhance fixation (i.e., osseointegration) between the structure and surrounding bodily structures, for example, to provide a matrix approximating natural cancellous bone or other bony structures. In this regard, a three-dimensional porous structure, or any region thereof, may be fabricated to virtually any desired density, porosity, pore shape, and pore size (e.g., pore diameter). Such structures therefore can be isotropic or anisotropic. Such structures can be infiltrated and coated with one or more coating materials. When coated with one or more biocompatible metals, any suitable metal may be used including any of those disclosed herein such as tantalum, titanium, a titanium alloy, cobalt chromium, cobalt chromium molybdenum, tantalum, a tantalum alloy, niobium, or alloys of tantalum and niobium with one another or with other metals. Illustratively, a three-dimensional porous structure may be fabricated to have a substantially uniform porosity, density, pore shape and/or void (pore) size throughout, or to comprise at least one of pore shape, pore size, porosity, and/or density being varied within the structure. For example, a three-dimensional porous structure to be infiltrated and coated may have a different pore shape, pore size and/or porosity at different regions, layers, and surfaces of the structure. According to certain embodiments of the present disclosure, regions of a three-dimensional porous structure to be infiltrated and coated may have a porosity as low as 55%, 65%, or 75% or as high as 80%, 85%, or 90%, or within any range defined between any pair of the foregoing values. In some embodiments, a non-porous or essentially non-porous base substrate will provide a foundation upon which a three-dimensional porous structure will be built and fused thereto using a selective laser sintering (SLS) or other additive manufacturing-type process. Such base substrates can comprise inner solid component 104 (FIG. 5), inner solid component 204 (FIG. 12), inner solid component 304 (FIG. 16) and inner solid component 404 (FIG. 20). Such base substrates can incorporate one or more of a variety of biocompatible metals such as titanium, a titanium alloy, cobalt chromium, cobalt chromium molybdenum, tantalum, or a tantalum alloy.

FIG. 26 is a schematic cross-sectional view of sleeve 700 of the present disclosure including additional functionality, including sensor module 702 and tubing system 704. Sleeve 700 can comprise body 706 having internal void 708. Body 706 can comprise any of sleeve 100, sleeve 200, sleeve 300 and sleeve 400 described herein. Treatment fluid 710 can be positioned within internal void 708. Sensor module 702 can comprise housing 712 in which sensor 714 and communications device 716 can be positioned. Internal void 708 can be provided with a divider, such as a wall of sleeve 700 to split internal void 708 into two chambers for receiving sensor module 702 and treatment fluid 710.

Sensor module 702 can comprise a device for obtaining information from anatomy surrounding sleeve 700. Sensor 714 can comprise a variety of different sensors, such as temperature, pH, force, vibration, impact, position, motion, capacitance, conductance, impedance, as well as other devices such as accelerometers, gyroscopes, thermometers, strain gages and the like. More than one sensor 714 can be included within housing 712. Sensor 714 can be positioned within housing 712 to abut or have access to an opening in housing 712 to facilitate engagement with anatomy. Thus, sleeve 700 can have a pocket or shelf to hold housing 712 within the confines of sleeve 700, but against an exterior wall of sleeve 700. For example, one side of housing 712 can be embedded in sleeve 700 while the opposite side is flush with the exterior of sleeve 700 to allow sensor 714 to be closer to anatomy to be sensed.

Communications device 716 can communicate using wireless communications signals, such as Bluetooth, Radio Frequency Identification (RFID), WiFi, Zigbee, infrared (IR), near field communication (NFC), 3GPP or other technologies. In examples, communications device 716 can communicate using one of more of the IEEE 802.15.6-2012 protocol, an MICS protocol and an MBANs protocol. In examples, communications device 716 can comprise a wired connection or can include a port for receiving a wire for a wired connection. Sensor 714 and communications device 716 can be located in housing 712, which can provide a barrier to biological matter from the surrounding anatomy and to treatment fluid 710.

Sensor module 702 can additionally comprise controllers, memory devices, circuit boards, processors, batteries, and antennas, as well as other electronics components.

Tubing system 704 can comprise a fluid conduit extending from internal void 708. Tubing system 704 can be utilized to fill or otherwise add treatment fluid to internal void 708. Tubing system 704 can comprise one or more lengths of tube or hose connected together to extend out of anatomy to which sleeve 700 has been implanted. Specifically tubing system 704 can penetrate through skin 718 to be accessible from outside the anatomy. Thus, tubing system 704 can be accessed postoperatively to initially add treatment fluid to sleeve 700 or to refill internal void 708 after initial treatment fluid has been depleted. Tubing system 704 can be configured similarly to or incorporated into a surgical drain system. A syringe or pump can be coupled to the free end of tubing system 704 located outside of skin 718 to push or force treatment fluid into internal void 708.

### VARIOUS NOTES & EXAMPLES

Example 1 is an anchoring component for implanting with a prosthetic component, the anchoring component comprising: an annular body comprising an interior surface defining a socket for receiving a stem of the prosthetic component and an exterior surface for engaging bone; an internal void within the annular body; and a functional component disposed within the internal void to provide supplemental functionality to the anchoring component.

In Example 2, the subject matter of Example 1 optionally includes wherein the functional component comprises a treatment fluid.

In Example 3, the subject matter of Example 2 optionally includes a passage extending into the exterior surface to intersect the internal void.

In Example 4, the subject matter of Example 3 optionally includes a plug positioned within the passage to limit or inhibit egress of fluid out of the internal void.

In Example 5, the subject matter of any one or more of Examples 2-4 optionally include wherein the treatment fluid comprises at least one of a drug, a biologics, a bone-growth promoting material, an antibiotic material, an infection preventing medium, a bone autograft material and a bone allograft material.

In Example 6, the subject matter of any one or more of Examples 2-5 optionally include a membrane extending over at least a portion of the internal void to prevent egress of the treatment fluid.

In Example 7, the subject matter of any one or more of Examples 2-6 optionally include a tube extending form the internal void to outside of the anchoring component.

In Example 8, the subject matter of any one or more of Examples 1-7 optionally include wherein the functional component comprises a sensor module.

In Example 9, the subject matter of Example 8 optionally includes wherein the sensor module comprises a sensor and a communications device.

In Example 10, the subject matter of any one or more of Examples 1-9 optionally include a plurality of internal voids.

In Example 11, the subject matter of any one or more of Examples 1-10 optionally include wherein the annular body comprises: an inner solid component; and an outer porous component.

In Example 12, the subject matter of Example 11 optionally includes wherein the inner solid component comprises: an inner surface defining the socket; and an outer surface; wherein the internal void is disposed between the inner surface and the outer surface.

In Example 13, the subject matter of Example 12 optionally includes wherein the internal void is bounded by material of the inner solid component.

In Example 14, the subject matter of any one or more of Examples 12-13 optionally include a passage extending from the exterior surface of the anchoring component to the internal void.

In Example 15, the subject matter of any one or more of Examples 12-14 optionally include wherein the internal void extends from an upper surface of the inner solid component to a lower surface of the inner solid component.

In Example 16, the subject matter of any one or more of Examples 11-15 optionally include wherein the inner solid component comprises: an inner surface defining the socket; and an outer surface; wherein the internal void extends into the outer surface to be in communication with outer porous component, the outer porous component having a porosity to allow fluid to pass therethrough.

In Example 17, the subject matter of Example 16 optionally includes a passage extending from the exterior surface of the anchoring component to the internal void through the inner solid component.

In Example 18, the subject matter of any one or more of Examples 16-17 optionally include an aperture extending from the exterior surface of the anchoring component to the internal void through the outer porous component.

In Example 19, the subject matter of any one or more of Examples 16-18 optionally include a central void at least partially circumscribing the socket between upper and lower surfaces of the inner solid component, the central void intersecting the internal void.

In Example 20, the subject matter of any one or more of Examples 1-19 optionally include wherein the anchoring component comprises a sleeve configured for positioning around a stem of a tibial implant, the exterior surface of the annular body comprising a posterior surface that is angle obliquely outward from an axis of the socket.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An anchoring component for implanting with a prosthetic component, the anchoring component comprising:
an annular body comprising an interior surface defining a socket for receiving a stem of the prosthetic component and an exterior surface for engaging bone;
an internal void within the annular body; and
a functional component disposed within the internal void to provide supplemental functionality to the anchoring component.

2. The anchoring component of claim 1, wherein the functional component comprises a treatment fluid.

3. The anchoring component of claim 2, further comprising a passage extending into the exterior surface to intersect the internal void.

4. The anchoring component of claim 3, further comprising a plug positioned within the passage to limit or inhibit egress of fluid out of the internal void.

5. The anchoring component of claim 2, wherein the treatment fluid comprises at least one of a drug, a biologics, a bone-growth promoting material, an antibiotic material, an infection preventing medium, a bone autograft material and a bone allograft material.

6. The anchoring component of claim 2, further comprising a membrane extending over at least a portion of the internal void to prevent egress of the treatment fluid.

7. The anchoring component of claim 2, further comprising a tube extending form the internal void to outside of the anchoring component.

8. The anchoring component of claim 1, wherein the functional component comprises a sensor module.

9. The anchoring component of claim 8, wherein the sensor module comprises a sensor and a communications device.

10. The anchoring component of claim 1, further comprising a plurality of internal voids.

11. The anchoring component of claim 1, wherein the annular body comprises:
an inner solid component; and
an outer porous component.

12. The anchoring component of claim 11, wherein the inner solid component comprises:
an inner surface defining the socket; and
an outer surface;
wherein the internal void is disposed between the inner surface and the outer surface.

13. The anchoring component of claim 12, wherein the internal void is bounded by material of the inner solid component.

14. The anchoring component of claim 12, further comprising a passage extending from the exterior surface of the anchoring component to the internal void.

15. The anchoring component of claim 12, wherein the internal void extends from an upper surface of the inner solid component to a lower surface of the inner solid component.

16. The anchoring component of claim 11, wherein the inner solid component comprises:
an inner surface defining the socket; and
an outer surface;
wherein the internal void extends into the outer surface to be in communication with outer porous component, the outer porous component having a porosity to allow fluid to pass therethrough.

17. The anchoring component of claim 16, further comprising a passage extending from the exterior surface of the anchoring component to the internal void through the inner solid component.

18. The anchoring component of claim 16, further comprising an aperture extending from the exterior surface of the anchoring component to the internal void through the outer porous component.

19. The anchoring component of claim 16, further comprising a central void at least partially circumscribing the socket between upper and lower surfaces of the inner solid component, the central void intersecting the internal void.

20. The anchoring component of claim 1, wherein the anchoring component comprises a sleeve configured for positioning around a stem of a tibial implant, the exterior surface of the annular body comprising a posterior surface that is angle obliquely outward from an axis of the socket.
